(19) United States
(12) Patent Application Publication
(10) Pub. No.: US 2012/0168641 A1
(43) Pub. Date: Jul. 5, 2012

(54) UV PTFE DIFFUSER TECHNOLOGY

(76) Inventor: Todd E. LIZOTTE, Hooksett, NH (US)

(21) Appl. No.: 13/228,016

(22) Filed: Sep. 8, 2011

Related U.S. Application Data
(60) Provisional application No. 61/380,779, filed on Sep. 8, 2010.

(51) Int. Cl. A61L 2/10; A23L 1/025; C02F 1/30; G21K 5/04; A61L 2/08

(52) U.S. Cl. 250/435; 250/503.1; 250/504 R

(57) ABSTRACT

A system for emitting diffused ultraviolet radiation for the disinfection of air, water, food or other surfaces includes a laser for generating a laser beam in a desired wavelength range and a beam shaping system. The beam shaping system includes a beamsplitting optical element for splitting the laser beam into a plurality of beamlets and at least one diffusive reflective element for reflecting at least one beamlet with a diffused radiation profile wherein the at least one diffusive reflective element comprises one of polytetrafluoroethylene (PTFE) and barium sulphate.

### CROSS REFERENCES TO RELATED APPLICATIONS

The present Application is related to and claims benefit of U.S. Provisional Patent Appln. No. 61/380,779 filed Sep. 8, 2010 by Todd E. LIZOTTE for UV PTFE DIFFUSER TECHNOLOGY.

### FIELD OF THE INVENTION

The present invention relates to the generation, shaping and distribution of UV radiation for purposes of disinfection of food, water, air as well as various surfaces and environments including quarantine facilities and high traffic volume areas and for the generation and shaping of laser generated general visible radiation illumination of areas and, in particular, to such systems employing polytetrafluoroethylene (PTFE) diffusion, reflecting and beam shaping elements.

### BACKGROUND OF THE INVENTION

It is well known and acknowledged by those familiar with past and present health issues, and in particular with disease control issues, that there is a continuing and growing need for effective, efficient, environmentally friendly and cost effective systems for the elimination of disease causing factors for a wide range of applications. Typical applications include, but are not limited to, the disinfection of food of all types, the provision of clean, safe water for a variety of uses, including drinking, medical and food, semiconductor and pharmaceutical manufacturing, and the disinfection of air and various surfaces in a wide range of environments, including quarantine and medical facilities and even in high traffic and congestion areas, such as buildings and airports in bioterrorist events or pandemics and epidemics.

Although chemical disinfection is possible and has often been used in systems for the disinfection of food, water, air and surfaces of all types, more recent efforts have recognized the effectiveness of ultraviolet (UV) radiation for purposes as deactivating and disinfecting airborne and surface resident viruses in, for example, hospitals, mass scale quarantine facilities, homes, high traffic and congestion areas, and for the disinfection of food and water.

It is well known and understood that infectious diseases, such as methicillin-resistant staphylococcus aureus (MRSA), tuberculosis, various flu and cold viruses and many other pathogenic microorganisms, including nosocomial infections, that is, infections originating in hospitals and other medical facilities, can be spread by airborne transmission, physical transport, such as on clothing, surgical instruments, table surfaces, door knobs, hand rails, and so on, and can be readily spread in water and in food.

It has also become well recognized, however, that UV is highly effective against a wide range of pathogenic microorganisms and has a great potential for destroying such microorganisms or at least reducing the spread of such microorganisms.

The more commonly encounter pathogenic microorganisms that are destroyed by UV radiation include, for example, but are not limited to, Bacillus anthracis, methicillin-resistant staphylococcus aureus (MRSA), Cryptosporidium, Corynebacterium diphtheriae, Dysentary bacilli (diarrhea), Escherichia coli (diarrhea), Legionella pneumophilia, Mycobacterium tuberculosis, Pseudomonas aeruginosa, Salmonella (food poisoning), Salmonella paratyphi (enteric fever), Salmonella typhosa (typhoid fever), Shigella dysentariae (dysentery), Shigella flexneri (dysentery), Staphylococcus epidermidis, Streptococcus faecaelis, Vibro commo (cholera), Bacteriophage (E. Coli), Hepatitus, Influenza, Poliovirus (poliomyelitis) and Baker's Yeast.

Therefore considering the nature of UV radiation and the means by which UV radiation destroys pathogenic microorganisms, UV radiation, also referred to as ultraviolet light, is electromagnetic radiation with a wavelength shorter than that of visible light. The spectrum consists of electromagnetic waves with frequencies higher than those that humans identify as the color violet (purple). As illustrated in FIG. 1, the UV radiation spectrum 10 is typically considered as consisting of various wavelength ranges, each having, to a certain extent, its own particular characteristics and uses, such as the UV-C range 10C from 100 nanometers (nm) to 280 nm, the UV-B range 10B from 280 nm to 315 nm, and the UV-A range 10A from 315 nm to 400 nm. FIG. 1 illustrates the electromagnetic spectrum and highlights the UV spectrum and the specific germicidal wavelengths of interest when considering the disinfection or purification of air, for example.

As indicated, UV-C radiation in the 100 nm to 300+ wavelength range is often referred to as “germicidal UV” range 10G, with optimum UV germicidal action occurring around 254-270 nm, and is typically used to neutralize the DNA of bacteria, viruses and other pathogens. UV radiation does so by destroying their ability to multiply, thereby defeating their ability to cause disease.

FIG. 2 illustrates a DNA chain 12A, not subjected to UV radiation, and a DNA chain 12B, subjected to UV radiation. The UV light penetrates through the cell wall and cytoplasmic membrane and causes a molecular rearrangement of the microorganism's DNA, which prevents the microorganism from reproducing. More specifically, UV-C light causes damage to the nucleic acid of the microorganisms by forming covalent bonds between certain adjacent bases in the DNA. The formation of such bonds prevents the DNA from being unzipped for replication, and the organism is unable to reproduce and, when the organism tries to replicate, it dies.

Considering the characteristics of UV radiation disinfection processes for disinfection of substances having high liquid content, such as water, food and beverages, the successful microbial disinfection of substances having a high liquid content requires the reliable eradication of all known spoilage microorganisms, including bacteria, viruses, yeasts and moulds and their spores, as well as other pathogenic organisms. Although traditional methods have employed chemical disinfection, such as chlorination, and physical disinfection, such as pasteurization, UV radiation disinfection has come to be recognized as one of the most promising methods to reach the regulatory standards for reduction of pathogens in food and liquids.

In this regard, UV disinfection is unique in that the UV radiation energy is absorbed by the fluid in which the microorganisms are suspended, leading to non-uniform fluence rates. The simplified form of the radiative transfer equation is Lambert-Beer's law,

Iₓ=I₀(−αx)
Where;
Ix, fluence rate at path length x, mW/cm2;
I0, incident fluence rate, mW/cm2;
α, absorbance coefficient (e base), cm−1;
x, path length, cm.

It can be seen, therefore, that the radiation fluence rate decreases exponentially with the path length from the radiation source. In other words, the non-uniform disinfection rates caused by the non-uniformity of fluence rate can severely limit disinfection efficiency especially when liquid foods with high absorption coefficients are treated, which is a factor which must be considered when designing UV disinfection systems.

UV disinfection is advantageous in food, water and beverage processing, however, since UV disinfection does not require the use of chemicals and, unlike chemical treatments, does not introduce any toxins or residues into processed water and does not alter the chemical composition, taste, odor or pH of the food or fluid being disinfected. UV disinfection is also versatile, capable of being deployed at the front and/or backend of a water, a beverage or a food processing line, and even incoming water, syrups and recycled water can be disinfected and re-introduced into the process. As indicated above and as will be discussed further below with regard to all UV disinfection process, a primary factor in the successful use of UV disinfection is the efficient illumination of the water, the food, the beverage or other item to be disinfected at levels appropriate to kill the microbiological contaminants.

Considering the specific case of UV radiation disinfection of water, such as drinking water and water used in various industrial processes, including the preparation of food products and pharmaceuticals, the more recently developed processes for disinfection of water include Advanced Oxidation Processes (AOPs), also referred to as photochemical synergistic oxidation processes, which target pollutants that are resistant to the standard methods of treatment. AOPs leverage the fundamental characteristics of UV light in which certain specific wavelengths are of sufficient energy to raise atoms or molecules to excited electronic states that are unstable. AOPs rely on one of two types of reactions, one being the transfer of energy when the atom or molecule drops from a high to lower energy state and the other being the transfer of photon energy into a chemical reaction which neutralizes the contaminant/pollutant. As with traditional UV water treatment, the critical aspect of the AOP process is the appropriate illumination or concentration of the UV energy to cover the water flowing through the reactor and to ensure that appropriate UV dosages are applied to generate the photochemical reactions. It has been specifically reported that due to variables including hydraulic residence, which is how fast the water moves through a system in equilibrium, the AOPs reaction time become dosage dependent. The time it takes for the chemical reaction to take place and neutralize the contaminants and may require a dosage ranging from 40,000 to 80,000 J/m². As indicated in the literature and by design with a majority of UV based processes, beam utilization or efficiency is critical and the ability to shape the UV light to efficiently illuminate the processing area offers superior process performance.

Current UV radiation treatment systems, however, including AOP systems, are disadvantageous in that they typically heavily rely on a technology that has not changed drastically over several decades. More specifically, the UV light is typically generated through the conversion of electrical energy in low pressure mercury vapor “hard glass” quartz lamps where electrons flow through ionized mercury vapor between the electrodes of the lamp, which then creates UV light within the range required for germicidal effectiveness. These types of lamps also generate IR radiation and broad spectrum UV, so that there are heating and aging issues, including solarization of the glass housings which, in turn, can reduce their effective lifetime. Another issue is that UV treatment systems, using low to medium pressure mercury lamps, typically employ a method of light delivery that is rather crude, using simplistic tubular window designs that are geared toward broadcasting the UV and other spectrums of radiation emitted from the lamp over a general area. Typical systems employ multiple lamps in staggered radial arrays, either across the flow or axial to the flow of the water, to achieve two objectives, namely, flood the water with UV and to create enough turbulence to increase the effectiveness of the UV exposure to the water.

In all cases, the designers need to absolutely ensure that the water is disinfected properly and, as a result, multiple lamps are typically utilized to provide levels of redundancy to thereby illuminate the intended treatment zone from various angles to ensure appropriate coverage. This process is illustrated in FIGS. 3A and 3B which show two common reactor configurations, each comprising a flow chamber 14A through which the water 14B flows and a plurality of UV sources 14C which irradiate the volume of water 14B flowing through the flow chambers 14A. Furthermore, the removal of suspended solids or particulate matter is important in order to ensure that the water treatment is adequate because this type of debris in the water can shield microbes from the UV light as they pass through the UV treatment system, which is an unacceptable situation with grave consequences when considering the treatment of drinking water or ultra pure water treatment facilities for medical and biomedical applications. UV treatment of water is, therefore, best used only after any sediment, turbidity and/or cloudiness has been adequately removed or reduced, such as by iron or manganese filtration or containment.

Considering the use of UV radiation to disinfect air and exposed surfaces, such UV radiation processes are advantageous as the processes are environmentally friendly and completely eliminate the need to handle or store dangerous chemicals and, because it is a flood process, there are no problems of overdosing or over exposure. Disinfection by UV radiation also has a low initial capital cost as well as reduced operating expenses when compared with similar technologies, such as ozone or air filter exchange systems. This is due in part to the fact that there is no lag in treatment of the air or the surfaces since the process is nearly instantaneous, without a need for holding plenums or long retention time requirements.

Again, however, current air and surface treatment systems heavily rely on the same basic technology as used in the disinfection of water, which as described above has not changed in several decades. As described, the UV radiation is generated through the conversion of electrical energy in low pressure mercury vapor “hard glass” quartz lamps where electrons flow through ionized mercury vapor, between the electrodes of the lamp, which then creates UV light within the range required for germicidal effectiveness. As also described above, these types of lamps also generate IR radiation and broad spectrum UV, resulting in heating and aging issues, including solarization of the glass housings, that can reduce their effective lifetime. Within an air handling environment, the conduction of heat from the tubes relies on a less efficient convection than occurs in water treatment systems and, with the resulting contrast in heating and cooling, it is possible for condensation to form, which can cause adverse conditions that might allow airborne contagions to escape, such as through drainage areas.

Another issue is that UV air treatment systems using low to medium pressure mercury lamps typically employ a method of light delivery that is again rather crude, using simplistic tubular window designs that are geared toward broadcasting the UV and other spectrums of radiation emitted from the lamp over a general area. Typical systems employ multiple lamps in staggered radial arrays, either across the flow or axial to the flow of the air, to flood the air with UV radiation and to create enough turbulence so as to increase the effectiveness of the air exposure to the UV radiation. As in the case of water treatment systems, the designers must absolutely ensure that the air is disinfected properly and, as a consequence, typically employ multiple lamps to provide levels of redundancy and thereby illuminate the intended treatment zone from various angles to ensure appropriate coverage.

The UV bulb or UV lamp systems of the prior art also suffer from a number of other clear disadvantages. For example, the design of a bulb/lamp system is constrained by various assumptions regarding the intensity of the UV output, or its profile. A lamp/bulb radiation intensity pattern can, however, be unpredictable from bulb to bulb or lamp to lamp, so that it is difficult to adequately model the radiation levels and profile to be obtained from a given design.

In many cases, particularly in flowing water or air disinfection systems, the designers have introduced turbidity into the water or air flow by mixing techniques to thereby increase the system disinfection efficiency. In a number of such systems, however, the radiation from the lamps or bulbs does not span the entire width or length of the duct, so that any assumption and use of complete mixing tends to result in larger errors compared to unmixed flow. The efficiency of such systems will tend to fall somewhere between those of systems which assume mixed flow and systems which assume unmixed flow, which limits the efficiency of such systems as well as the accuracy by which the efficiency of such systems may be evaluated, thereby requiring at least the use of redundant lamps or bulbs in order to ensure a minimum acceptable level of efficiency.

The use of UV radiation reflectors and radiation profile shaping elements can improve performance and reflectivity and can be an economical way of intensifying the Ultraviolet Germicidal Irradiation (UVGI) field in, for example, an enclosed duct or chamber as long as the reflectivity of the reflectors and profile shaping elements is selected in conjunction with and designed with a stable and predictable UV source. As will be discussed in following description of the present invention, most materials possess a combination of specular and diffuse properties and exhibit a degree of directional reflective dependence. The problem with current duct systems is, however, that the diffuser properties are not predictable and produce intensity variations, creating further problems with modeling the disinfection capability or efficiency of the system.

In other problems related to UVGI design processes, it must be noted that the reflective properties of various materials vary with the type, that is, the wavelength of the radiation impinging on the materials, and have other characteristics effecting their suitability for use in UV disinfection systems. For example, some materials reflect visible light, but not UV light. Polished aluminum is highly reflective of UV wavelengths, but is susceptible to oxidation, while copper, which reflects most visible light, is transparent in the UV range, and galvanized steel, which is typically used in HVAC (heating, ventilating and air conditioning) duct systems, has poor reflectivity in the UV range.

In other related problems, it must be noted that there presently exists no simple method of calculating the three-dimensional UVGI intensity field for specular or diffuse reflectors for lamp or bulb UV sources. Ray-tracing routines, using Monte Carlo techniques, are one approach, but the results do not easily lend themselves to analysis.

The present invention provides a solution to these and other related problems of the prior art.

### SUMMARY OF THE INVENTION

The present invention is directed to a system for emitting diffused radiation and includes a radiation source for generating an illuminating beam in a desired wavelength range and a beam shaping system wherein the beam shaping system includes a beamsplitting optical element for splitting the illuminating beam into a plurality of beamlets and at least one diffusive reflective element for reflecting at least one beamlet with a diffused radiation profile wherein the at least one diffusive reflective element comprises either polytetrafluoroethylene (PTFE) or barium sulphate.

In present embodiments of the system, the emitted radiation is in an ultraviolet wavelength range and preferably within a 100 nm to 300+ nm germicidal wavelength range.

The system of the present invention for emitting diffused radiation may further include a treatment chamber for accommodating a flow of one of a liquid and a gas wherein the at least one diffusive reflective element is arranged to distribute the diffused radiation profile within a volume of the treatment chamber.

In at least certain embodiments of the present invention the liquid may be water or a food containing water and the gas may be air.

In further embodiments of the invention the beamshaping system may further include at least one second diffusive reflective for reflecting the at least one beamlet with a diffused radiation profile with a further diffused radiation profile.

In further embodiments of the invention the radiation source may be any of a laser, a solid state light emitting diode or a lamp emitting in the desired wavelength range.

In yet other embodiments of the invention at least one PTFE diffusive reflective element may further include a wavelength shifting additive to allow shifting of the wavelength emitted by the radiation source to wavelengths different form the wavelengths emitted by the radiation source.

In other embodiments of the invention the at least one diffusive reflective element for diffusively reflecting the at least one beamlet with a diffused radiation profile may comprise at least one interior surface of the treatment chamber.

### BRIEF DESCRIPTION OF THE DRAWINGS

FIG. 1 is a diagrammatic illustration of the UV electromagnetic spectrum;

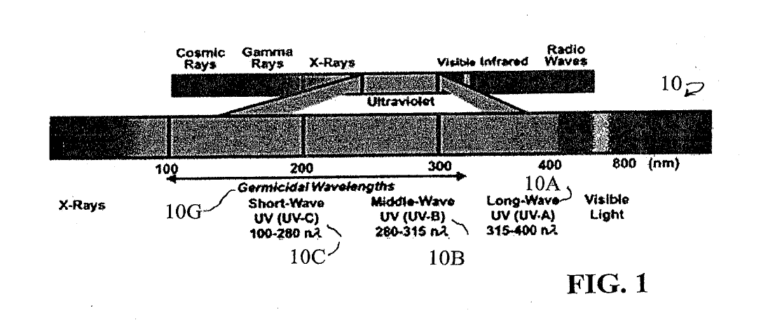

FIG. 2 is a diagrammatic illustration of the effects of UV radiation on pathogenic microorganisms;

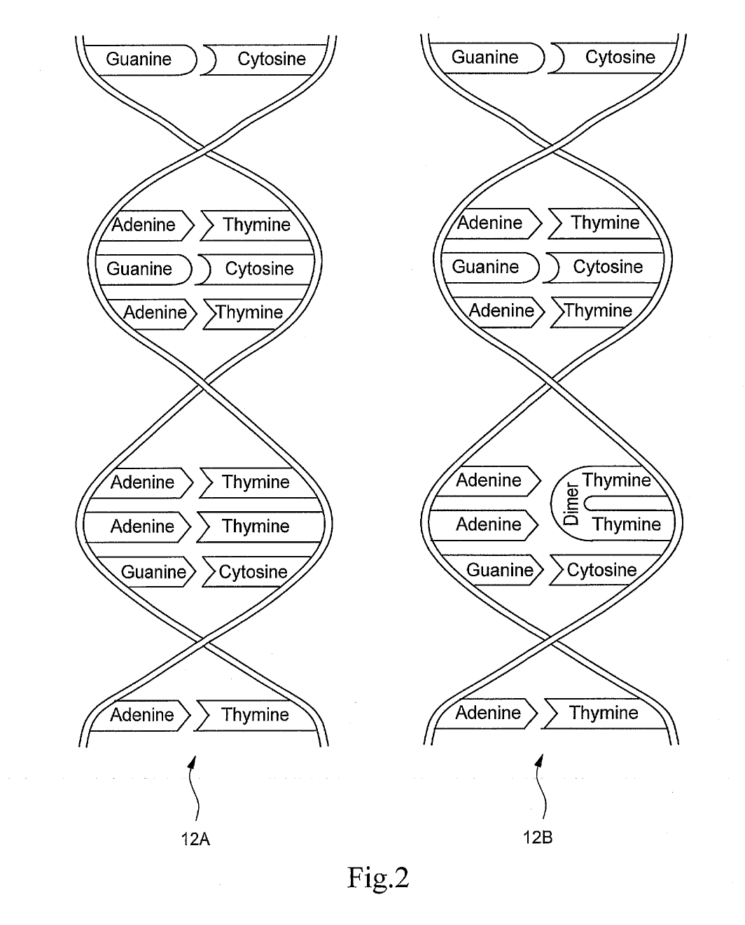

FIGS. 3A and 3B are diagrammatic illustrations of known UV radiation water purification systems;

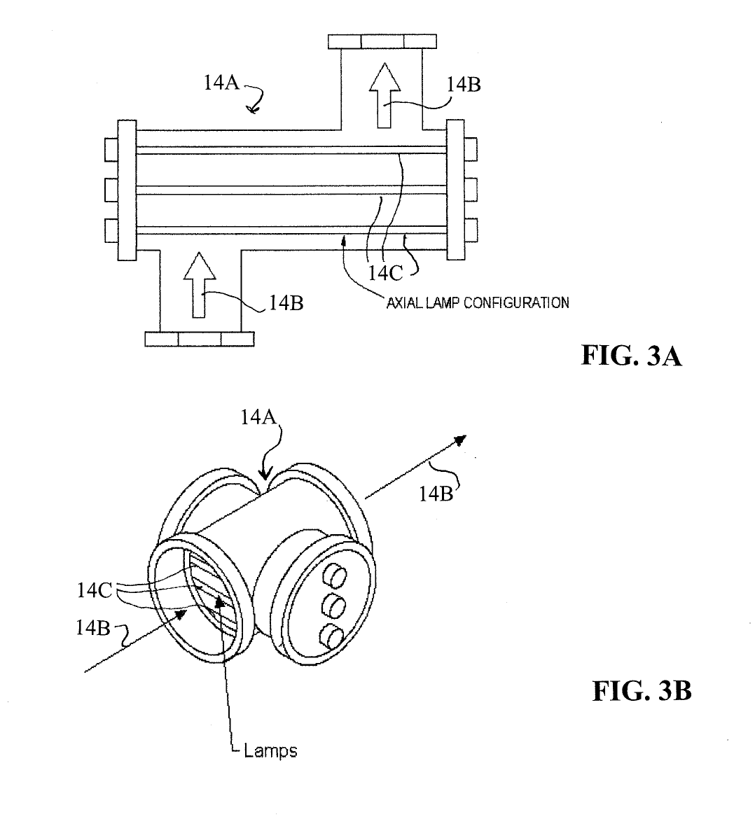

FIGS. 4A and 4B are diagrammatic illustrations of the use of optical elements to shape a laser beam;

FIGS. 4C and 4D respectively diagrammatic illustrations of a conically shaped Lambertian diffuser and faceted pyramidal shape Lambertian diffuser;

FIG. 4E is a diagrammatic view of a longitudinal illuminated diffuser using diffuse reflection and beam shaping optics;

FIGS. 5A-5D are diagrammatic views of PTFE blocks as Lambertian diffusers;

FIGS. 5E and 5F are diagrammatic views of specular and diffuse reflection;

FIGS. 6A-6C are diagrammatic views of UV water treatment systems; and

FIGS. 7A and 7B are diagrammatic views of a UC air treatment system.

### DETAILED DESCRIPTION OF THE INVENTION

The following will describe aspects of the present invention, including, and in that order, sources for the generation of UV radiation in the germicidal wavelengths, designs and design considerations for optical beam shaping elements for forming UV radiation from a laser UV generating device into a UV radiation profile suitable and appropriate for microorganism elimination from, for example, air, food and water or other liquids, methods for fabrication of optical beam forming elements according to the present invention, and exemplary designs of UV radiation disinfection systems according to the present invention.

A. UV Radiation Sources

First considering sources of UV radiation suitable for disinfection systems of the present invention, presently preferred sources of UV radiation, for purposes of disinfection of air, water or food or of surfaces in general include, one of the more significant parameters in the selection of a laser source is the wavelength of the radiation emitted by the laser, which is often primarily a matter of selection of the materials from which the laser is fabricated. Gas and solid state lasers, suitable for these purposes, are available with varying lasing properties and are well known to those of ordinary skill in the relevant arts. It is also well known that it is possible to tailor the output wavelength of the laser sources, for example, to fit the requirements of air or water disinfection, by changing the gas mix or by use of harmonic shifting crystals. Lasers of interest include, excimer lasers, which can e filled with KrC and KrF gas chemistry that produce wavelengths of 222 nm and 248 nm, respectively. Also of interest are solid state lasers, such as Diode Pumped Solid State (DPSS) lasers, which be configured with various lasing rod materials, such as, for example, Nd: YAG, Nd: YLF, Nd: YLG, Nd: YVO4, Ti: Sapphire and Alexandrite. All of these solid state lasers produce IR wavelengths that can be shifted to ultraviolet wavelengths ranging between 180 nm and 355 nm, that is, to the 3^{rd}, 4^{th} and 5^{th} harmonics, by the use of harmonic crystals such as LBO, BBO, KTP and CLBO.

Other sources of UV radiation that are of interest, according to the present invention, include solid state UV sources such as light emitting solid state devices (SSDs), which offer significant advantages, including reduced operating costs, increased safety, reliability, quality and stable output intensities. As is well known and understood by those of ordinary skill in the relevant arts, SSDs comprise a pn junction formed by two dissimilarly doped semiconductors. By applying an external electric field across the junction, current can be made to flow, and when the holes from the p-type and electrons from the n-type meet at the junction, they combine and release a photon of light wherein the wavelength of the emitted light depends on the bandgap energy of the materials used in the pn junction. The benefit of solid state UV sources is their life span, which may exceed 10,000 hours, while maintaining consistency and a narrow band of around 40 nm typical. The biggest issue facing SSDs is that the wavelength is limited to a peak around 395 nm for commercially available units. It is possible to customize the pn junction to shift to lower wavelengths, such as the 253.7 nm that is generally optimum for disinfection purposes, at a possible increased cost.

B. Design Considerations For Optical Beam Shaping Elements

Next considering designs and design considerations for optical beam shaping elements for forming UV radiation from a laser UV generating device into a desired UV radiation profile, it is well known that UV radiation sources emit UV radiation in the form of a tightly formed coherent beam. The disinfection of a volume of air, food, water or other liquid or various surfaces requires, however, that the beam emitted by the UV source be formed into an appropriate distribution profile so that the volume to be treated is reliably irradiated at levels sufficient to ensure the complete elimination of any microorganisms contained therein or thereon.

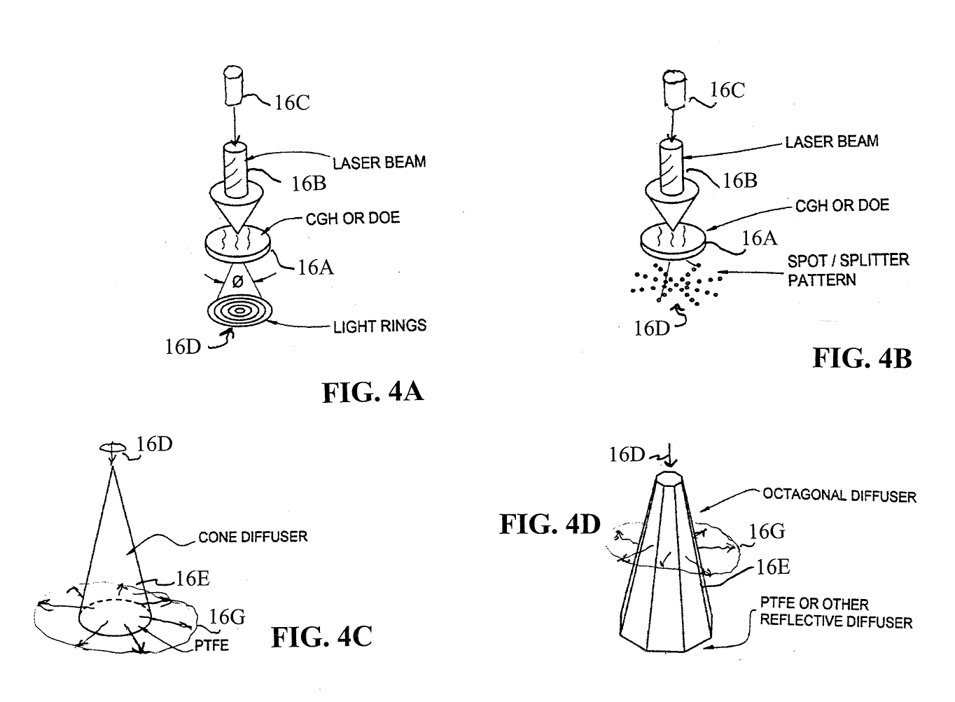

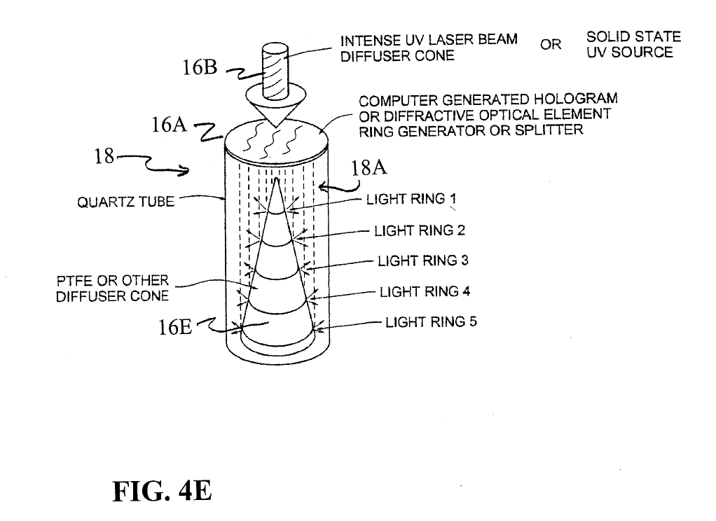

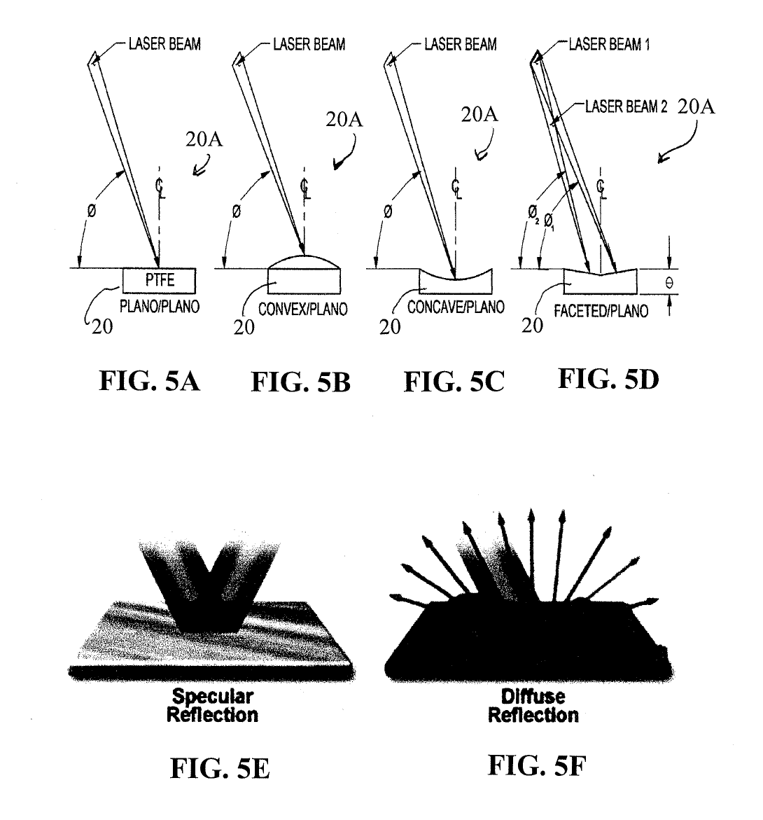

Exemplary embodiments of optical beam forming elements suitable for these purposes, according to the present invention, are illustrated in FIGS. 4A-4F. It will be understood, however, that possible embodiments of such optical beam forming elements are not limited to those specifically illustrated in FIGS. 4A-4F, and that other possible embodiments will be readily apparent and understood by those of ordinary skill in the relevant arts.

Referring now to FIGS. 4A and 4B, therein are respectively illustrated the use of an optical element 16A to shape a laser beam 16B emitted by a laser source 16C to provide a UV radiation pattern 16D having a profile dependent upon the specific design of the respective optical element 16A. In the exemplary embodiments shown therein, for example, the optical elements 16A may comprise computer generated holograms (CGHs) or diffractive optical elements (DOEs), both of which are well known and understood in the relevant arts, and the resulting radiation profiles may be, for example, light rings as illustrated in FIG. 4A or a spot/splinter pattern as illustrated in FIG. 4B. It will be understood by those of ordinary skill in the relevant arts, however, that other types and kinds optical elements 16A and other radiation profiles 16D may be employed, without departing from the spirit and scope of the present invention.

It must also be noted that in this embodiment of the present invention, as well as all other embodiments of the present invention described herein, the laser source 16C or other laser source employed in the present invention may be comprised, for example, of a laser, a solid state UV source such as a light emitting diode or any other form of radiation source, such as a conventional lamp or bulb, in a desired wavelength range ranging from the UV wavelengths to visible light wavelengths.

Once shaped by an optical element 16A, the resulting radiation profile 16D may then be passed through a diffusing element 16E comprising, for example, a conically shaped Lambertian diffuser 16E, as illustrated in FIG. 4C, or a faceted pyramidal shape Lambertian diffuser 16E, as illustrated in FIG. 4D, or a Lambertian surface diffuser, which will be described below, to diffuse the radiation profile 16D into a desired relatively even distribution radiation profile 16G which is then used to irradiate the volume of air, water, liquid or food to be disinfected.

Next referring to FIG. 4E, therein is shown an embodiment of a longitudinal illuminated diffuser (LID) 18 using both a diffuse reflection and beam shaping optics and comprising, according to the present invention, optical shaping and diffusing elements, as illustrated in FIGS. 4A-4D, but fabricated of polytetraflouroethylene (PTFE) in accordance with the present invention.

As illustrated in that Figure, the LID 18 comprises a tubular assembly in which a laser beam 16B is directed into a first end of the tubular assembly and through an optical element 16A comprising, for example, a computer generated diffractive splitter, such as a DOE, or some other diffractive shaper, such as a CGH, to spit the laser beam 16B into a radiation profile 16D comprising a plurality of beamlets 18A comprising, for example, of circular rings, as illustrated in FIG. 4A, or spot beamlets, as illustrated in FIG. 4B, or some other geometric shape(s). As each of the beamlets 18A cascades downward through the LID 18, they illuminate various points along the surface the diffuser 16E comprising, for example and as described above, a conically shaped Lambertian PTFE diffuser 16F or a faceted pyramidal shaped Lambertian PTFE diffuser 16F. As the beamlets 18A strike the surface of the diffuser 16F, they each are reflected from the surface of the diffuser 16F to diffuse in a Lambertian reflectance pattern thereby creating a pseudo-uniform circumferential illumination 16G along the length of the tubular assembly, which is typically encased in a quartz tube. It will thus be understood that the LID 18 of the present invention comprises a compact tubular structure providing UV disinfection source that can be placed within a centrifugal reactor or a pipe based reactor chamber.

C. Materials For UV Beam Forming Elements (PTFE)

The above descriptions of the present invention state that certain elements of a UV radiation and distribution system of the present invention, such as the LID 18 that may be employed in a water, food, liquid or air disinfection system, comprises Lambertian diffusers that, in turn, comprise material identified as polytetraflouroethylene (PTFE). The following descriptions will therefore next describe and discuss these aspects of the present invention, first considering the use of polytetraflouroethylene (PTFE) as a material for the fabrication of optical elements, such as diffusers in a UV laser system, and then considering diffuse and specular reflectance as implemented in a system according to of the present invention.

PTFE is one of the few known materials that does not absorb water, is almost completely chemically inert with respect to a wide variety of industrial chemicals, such as strong acids, alkalis or oxidants, and maintains these properties at elevated temperatures and pressures. These properties occur due to strong inter-atomic bonds between the carbon-carbon and carbon-fluorine atoms of PTFE, with an almost perfect shielding of the carbon backbone by the fluorine atoms and a high molecular weight. While nearly all plastics absorb small quantities of certain materials they come in contact with, there is essentially no chemical reaction between PTFE and most other substances. The high molecular weight reduces the number of microscopic voids between the molecules which provide space for foreign substance to be lodged. PTFE resins absorb practically no common acids or bases at temperatures as high as 200° C., even over extended exposures. In certain circumstances, because of PTFE properties, additives can be compounded to the PTFE to provide further fabrication options, such as bonding, although these compounds, such as FEP (fluoronated ethylene propylene), may reduce the effectiveness of the PTFE product once assembled. PTFE may also be formed by PTFE isostatic molding techniques, which offers the benefit of a molding technique for fabricating large, complex assemblies on a macro level and through a patented process and allows the fabrication of laser system optical elements that replicate complex reflective and diffusing micro optical structures to enhance the natural reflective diffuse nature of PTFE materials.

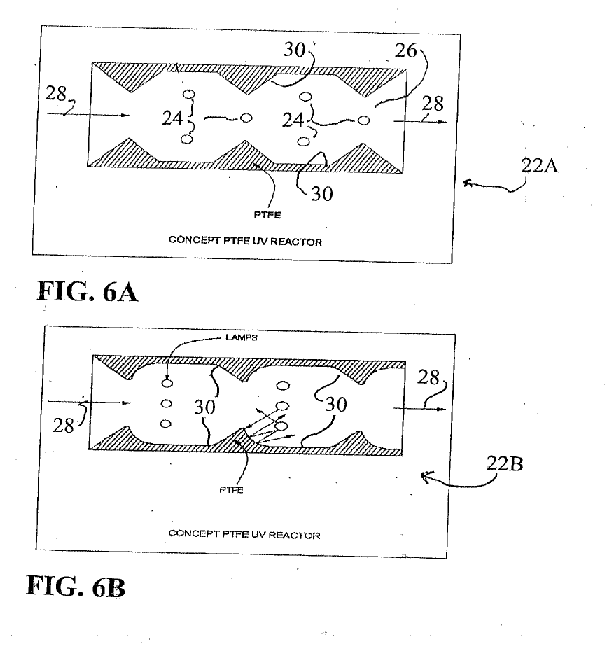

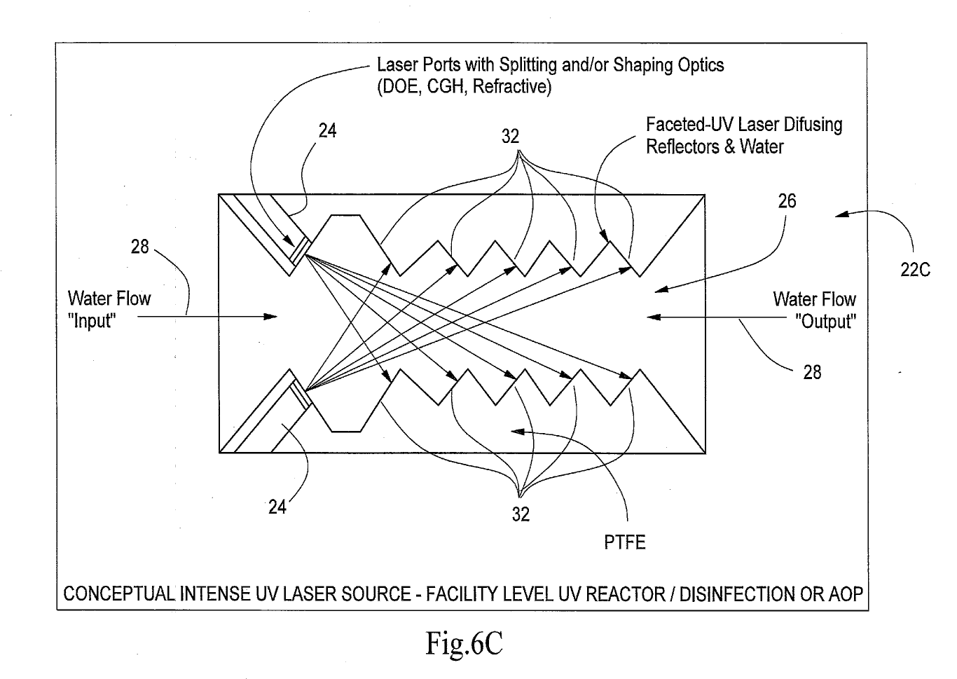

FIGS. 5A-5D, for example, are diagrammatic illustrations of the use of PTFE blocks 20, as Lambertian diffusers 20A, having a range of diffusion angles.

D. Specular and Diffuse Reflection and Lambertian Surfaces

Turning now to and considering diffuse and specular reflectance and the use of Lambertian surfaces as diffusion elements in a UV laser system, FIGS. 5E and 5F are respectively diagrammatic illustrations of specular reflection and diffuse reflection. A specular or mirror-like reflection surface, as illustrated in FIG. 5E, reflects radiation impinging on the surface at an angle equal to the angle at which the radiation arrives and impinges on the surface. A Lambertian surface, however, as illustrated in FIG. 5F, reflects light or radiation impinging on the surface with equal radiance or luminance in all directions, that is, scatters the impinging radiation over a wide range of directions regardless of the angle or angles at which the radiation impinges on the surface. As a result, a Lambertian surface may thereby function as a virtual light source for radiating light or radiation evenly over a wide range of angles. Compressed PTFE materials have been known to provide near Lambertian performance when compared to the ideal, as may be shown by a comparison of compressed PTFE surfaces with specular reflection surfaces. In the case of compressed PTFE materials, the specular reflectance is >˜0.5% while its diffuse reflectance is <˜97%, over a wavelength range of 230 nm to 1080 nm, with ˜99% diffuse reflectance between 420 nm to 850 nm, which is virtually the reverse of specular reflection surfaces. Accordingly, and according to the present invention, the PTFE material may be formed, using for example vacuum micro forming/molding techniques, into useful reflective beam shaping elements to improve the efficiency of traditional and laser based UV reactor disinfection systems.

E. Fabrication of PTFE Beam Shaping Elements

Next considering the shaping of PTFE materials into UV laser reflective beam shaping elements according to the present invention, standard isostatic molding, bonding or forming processes rely on the assembly of parts or joints which are then subjected to isostatic or directional pressure, while simultaneously elevating the parts to its fusion temperature and maintaining this pressure through to the solidification phase forming an integral monolithic structure of pure PTFE. One such material is a product called Spectralon®, which is a thermoplastic resin consisting of PTFE material that is formed and can be machined into a wide variety of different shapes for the fabrication of optical components. Spectralon® gives the highest diffuse reflectance of any known material or coating over the UV-VIS-NIR region of the spectrum. The reflectance is generally >99% over a range from 400 nm to 1500 nm and >95% from 250 nm to 2500 nm and is resistant to UV degradation with NIST traceable calibration.

In a presently preferred embodiment of the present invention, the PTFE material is formed into a block material that has a void volume between 30% and 50% and is sintered into a relatively hard cohesive block so as to maintain such a void volume. In the case of micro forming or micro embossing micro optical forms or micro optics diffractive or diffusing structures, the void volume needs to be adjusted as a ratio of the particle size, such that the particles of PTFE or compounds of other additive materials can form the micro optical structures required to adequately provide an optical response when illuminated, that is, diffraction, reflection and/or diffusion of light can occur.

There are several methods used to form monolithic blocks of the material, however most of these techniques, when used to form surface micro-structures, suffer from the fact that air is trapped at the mold-material plane during the compression-embossing process and limits the ability of the material to resolve the required micron sized features or critical dimensions that effect the optical properties or function of the optical micro surfaces. As for the mold design itself there a number of configurations that are used to create an isostatic micro structure molding tool which can achieve the pressure required to form the desired component. Certain techniques utilize the differential thermal expansion between the PTFE and the mold materials, such as aluminum. Further compression methods include using Gutenberg printing press style of compression, where a rectangular box has a side that is pressed into the bulk material by an anvil that is screwed downward to form generic blocks of material.

Considering the relative characteristics of PTFE and aluminum in the use of differential expansion in forming objects such as UV optical elements from PTFE, PTFE has a linear coefficient of expansion of 5.4×10⁻⁵ in/in/° F. while aluminum has a linear coefficient of expansion of 14.4×10⁻⁶ in/in/° F. Taking these two materials as an example, when comparing PTFE and similar fluoro-polymers, they undergo about a four fold degree of expansion greater than that of aluminum, and it is the force of differential expansion which is utilized to bond PTFE to itself, to forge or transform the material into the desired part. This allows the pressure to be achieved without having to use active pressure via a hydraulic press. This type of mold is then either placed into a furnace or dunked into a molten bath of salt to raise the temperature quickly. Once the pressure and temperature for fusion has been achieved the mold is then quenched into water or left to cool. This quenching allows the mold to be quickly cooled and sufficiently shocks the part to allow it to release from the mold easily. Various compression techniques can also employ break away molds that can simply be pulled a part. The problem with most traditional methods is that they are not suited for achieving dimensional resolutions that require micro level tolerances.

A present embodiment of the techniques used to fabricate beam shaping elements of the present invention utilizes a compression mold designed to compress raw powder or pelletized PTFE material in conjunction with the application of vacuum to the mold cavity. The mold itself has a series of small vacuum ports to allow the mold to be placed under a vacuum to remove all the air and allow better compression and densification and reduce the chance for bubbles or large voids that could create discontinuities in the density or void volume that adversely effect the optical properties of the beam shaping elements defined by the microstructure being formed. Once the vacuum gauge achieves about <35 Torr, it is considered to be under low to medium vacuum. At this point, the mold anvil is bolted down to apply compression force and was placed into a high temperature oven. The mold is then elevated to a temperature of between 175° C. to 375° Co achieve its fusion/sintered temperature, which is based on the compounds being sintered and the reflective optical properties desired of the materials surface and the dimensional resolution required maintaining the critical dimensions of the microstructures. After reaching its critical temperature, the mold is allowed to sit for approximately 10 to 40 minutes, depending on the compound. Finally, the mold is removed from the oven and the mold is quenched in water and left to cool, after which the mold is opened to retrieve the part.

However, depending on the application and the optical properties required, the molding process can consist of a mold compression and heating cycle, without a quenching requirement, based on the mold tooling and the complexity of the part. An alternate process uses vacuum evacuation, wherein the material is placed under partial vacuum and the material is then compressed within a mold which holds the micro structured shims and or diamond machined inserts. Once compressed, the material is pre-sintered in a furnace for about 15 to 30 minutes and then allowed to cool. The resulting pre-sintered block is transferred to a carrier substrate and sintered at atmospheric pressure. The use of the vacuum offers the opportunity for better density uniformity and void control.

F. Further Aspects Of The Use of PTFE Optical Elements in UV Systems

In a further aspect of the use of PTFE optical elements for a UV disinfection system, it is noted that standard UV reactor chambers of the prior art do not typically utilize optical surfaces to enhance the dispersion or provide for retro reflection of the radiation once it is emitted from the lamps. For simple designs the critical parameter is the dose level at the wall surface of the reactor, which may be represented by the following approximation for the effective dose in a single axial lamp based UV reactor. According to this approximation,

[It]=Exposure Dose (D)=L*I*T=Io*T*L*(rₑ/rᵢ)*10^{-A(r}^{ₑ}^{-r}^{ᵢ}⁾
Where:
It=potentially biocidal UV dose
S=maximum irradiation surface (m²); A=2πrₑL; (L=length of reactor)
T=irradiation time (seconds)
A=absorbance
L=length of cylindrical reactor
rᵢ=radius of lamp+lamp enclosure, and
rₑ=(internal) radius of the cylindrical reactor.

It may be seen from the above example that in, for example, a UV water treatment system, the use of a retro reflective structure that can also act as a means of developing turbulence would enhance the process of water treatment by, for example, providing further chances for the UV to interact with the water flow. The concept of salvaging or reclaiming scattered emitted UV radiation by use of a retro reflective structure would thereby provide further opportunities for the UV radiation to interact with the water being treated.

This principle may be extended to yet other UV treatment systems, such as air and area or volume treatment systems by the providing of PTFE based reflective diffuse/specular faceted structures to create a structure that could be used to recover or reclaim or diffuse wasted UV light within a UV reactor by shaping the lamp or laser radiation or to diffuse the scattered UV striking the walls to enhance the efficiency of the UV reactor and its process.

Because of the previously discussed and well established spectral reflection limitations, a DOE optical element would require an illumination incident angle range of between 5° and 20° which would limit its efficiency and flexibility as a shaping tool, but not eliminate its possible use in, for example, a faceted mirror array shaper. Even with the possible reduction in efficiency, that is, total power reflected by a faceted mirror array shaper, it is possible, due to the nature of the UV reactor designs, that the smaller fraction of the reflected light still could provide or redirect enough of the UV light to improve the process.

In conclusion, therefore, and according to the present invention, laser based UV disinfection of air and water using standard PTFE substrate material for optical elements therein can be advantageous in UV reactor technology. As may be seen from the above discussions, since UV sources tend to produce a very coherent and small sized beam, the problem faced by designers is how that beam is shaped and delivered into the air or the water flow for maximum exposure for disinfection. Since PTFE material is a near perfect Lambertian reflector, it is possible, as described herein above, to split a laser beam with a diffractive optics into a series of smaller beams that can be dispersed across the flow area.

G. Exemplary Embodiments of UV/PTFE Systems

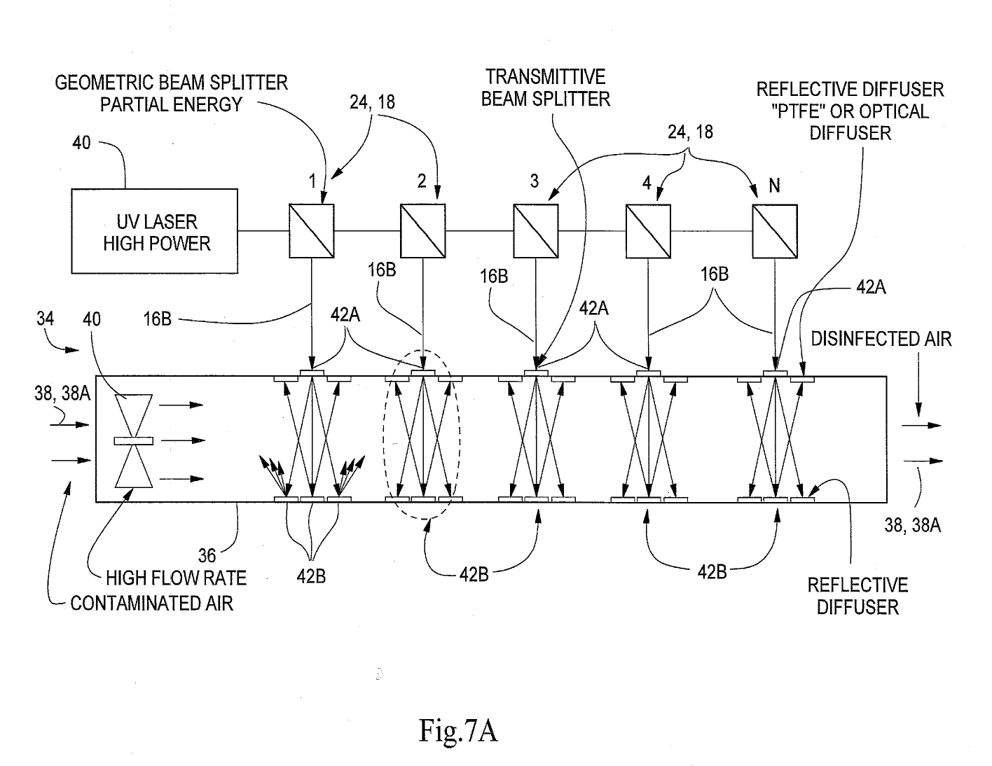

Referring to FIGS. 6A-6C, therein are shown diagrammatic illustrations various embodiments of UV water treatment systems 22A, 22B and 22C of the present invention. As indicated, the water treatment systems 22A and 22B employ a plurality of UV sources 24, such as LIDs 18, to irradiate the volume of a treatment chamber 26 through which the water 28 flows. As indicated, at least the interior surfaces of treatment chamber 26 comprise PTFE to form reflective optical surfaces 30 to diffuse the UV radiation throughout the volume of the treatment chamber 26.

The water treatment system 22C is similar to the systems 22A and 22B, except that the treatment chamber 26 is provided with two LIDs 18, or similar sources emitting beams of UV radiation 32, which are in turn reflected throughout the interior volume of the treatment chamber 26 by reflecting facets 32 formed of PTFE on the interior surfaces of the treatment chamber 26.

As discussed herein above, there are several key parameters that dictate how a UV reactor will be designed and operate, the most critical aspect of which is characteristics of the laser or lamp source 16C, such as the wavelength, the operational parameters, the orientation within the flow, and the radiation source monitoring, followed by general hydraulic conditions such as the flow pattern, the turbulence and the flow rate, as well as other parameters. The embodiment of the water treatment system 22A, 22B or 22C, according to the present invention, employs a PTFE molded structure, that is, the interior surfaces of treatment chambers 26, to provide enhanced operational capability to the UV reactor by providing retro-reflective capabilities or properties along the fluid path. Generically the molded PTFE could take the form of a simple diffuse/specular reflector with geometry to induce turbulence in recognition of the fact that the formation of turbulence is a critical condition for UV reactor based systems. Researchers have established that the water flow 28 in the chamber 26 must be turbulent and have a Reynolds numbers ≧2000 to ensure appropriate mixing and distribution of particulate within the flowing water being treated, and Reynolds numbers are commonly used by those of skill in the relevant arts to characterize different flow regimes, such as laminar or turbulent flow. Laminar flow occurs at low Reynolds numbers, where viscous forces are dominant, and is characterized by smooth, constant fluid motion, while turbulent flow occurs at high Reynolds numbers and is dominated by inertial forces, which tend to produce random eddies, vortices and other flow fluctuations.

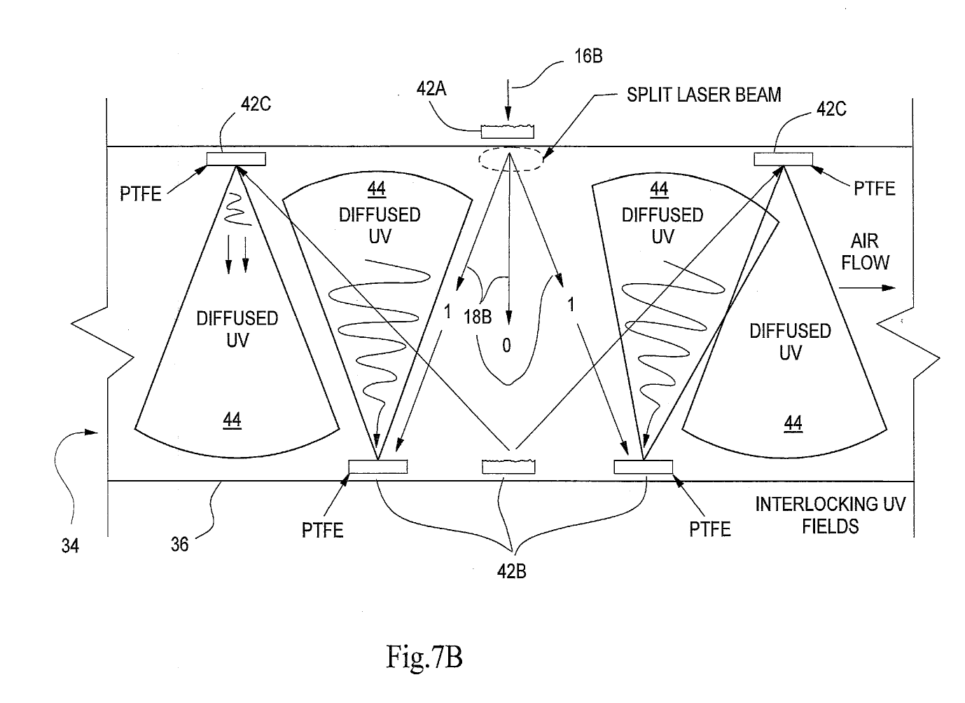

Referring now to FIGS. 7A and 7B, therein are respectively shown a diagrammatic view of a UV air treatment system 34 using UV sources and optical elements of the present invention in a turbulent air flow system, and a detailed diagrammatic view of a section of the UV air treatment system 34.

As shown therein, an air treatment system 34 of the present invention includes an air flow chamber 36 through which air 38 flows with contaminated air 38A entering a first end of the chamber 36 and exiting from the chamber as disinfected air 38B, with air 38 typically being driven by a fan system 40. The system 34 will include a plurality of UV radiation sources 24, such as lasers 16C as shown in FIGS. 4A-4E, spaced or located along a length of the chamber 36 with the UV radiation sources 24 typically being powered from a system power supply 40, which may be a general facility power supply or, in certain circumstances such as a temporary quarantine facility, may be dedicated to the system 34.

As indicated, each radiation source 24 emits a laser beam 16B into a corresponding entry port of the chamber 26 through a beamsplitter element 42A, which will typically comprise a transmittive beam splitter such as a CGH or DOE element, similar to elements 16A of the LID 18 illustrated in FIGS. 4A-4D. As indicated in FIGS. 7A and 7B, each of beamsplitter elements 42A result in a plurality of beamlets 18A being transmitted to the opposite wall of the chamber 36, where each beamlet 18A will impinge upon and be reflected and diffused from a first diffusive reflective element 42B, which may comprise, for example, Lambertian diffusers 20A comprised of PTFE blocks 20. As also indicated, the diffused beamlets 18A are reflected from diffusive reflective elements 42B and back to the first wall of the chamber 36, where they are again diffusively reflected from second diffusive reflective elements 42C, which again may comprise Lambertian diffusers 20A comprised of PTFE blocks 20.

As may be seen from FIGS. 7A and 7B, the result of the UV laser optical arrangement of an air flow chamber 34 of the present invention is that the air 38 flowing through the chamber 36 is irradiated throughout by diffused UV radiation profiles 44, thereby ensuring that the entire volume of air 38 moving through the chamber 36 is sufficiently irradiated so as to adequately disinfect the air 38.

Lastly, it will be seen from the above discussions of UV laser optical systems that the fundamental design discussed and described herein above may be readily adapted to the UV irradiation of surfaces as well as volumes. For example, a single LID 18 or cluster or array of LIDs 18 may be arranged and used to irradiate a stationary or moving surface or may be implemented in a hand held unit for portable use. In a like manner, the basic configuration of, for example, FIGS. 7A and 7B may be adapted to irradiate a surface by providing ports in the second wall of the chamber 36, that is, the wall on which first diffusive reflective elements 42B are mounted, to permit the diffused UV radiation to be emitted through the ports and onto any selected surface, such as the interior of a room.

Finally, it will be appreciated that while the present invention has been described as implemented in systems for the UV disinfection of water, air, food and other liquids, the system may also be adapted to the generation and distribution of general illumination radiation, that is, light in the visible range, to provide a source of visible illumination, by appropriate selection of the laser sources 16C and their emitted spectrums.

Further in this regard, it must be noted that the PTFE diffusive reflective element may further include a wavelength shifting additive, which may, for example, be added to the PTFE or used to treat or coat the PTFE diffusive reflective element to allow shifting of the wavelength emitted by the radiation source, that is, the laser, solid state light emitting diode or other lamp light source, to wavelengths different form the wavelengths emitted by the radiation source, such as from the UV wavelengths to the visible light wavelengths, such as white light. Examples of such wavelength shifting additives may include, for example, phosphor, as well as other substances or compounds well known to those of ordinary skill in the relevant arts.

It must also be noted that in alternate embodiments the diffusive reflective elements may be comprised of Barium “sulfate” is an alternative to PTFE, recognizing that PTFE provides better performance for wavelengths <400 nm.

Therefore, since certain changes may be made in the above described system and methods of the present invention without departing from the spirit and scope of the invention herein involved, it is intended that all of the subject matter of the above description or shown in the accompanying drawings shall be interpreted merely as examples illustrating the inventive concept herein and shall not be construed as limiting the invention.

What is claimed is:

1. A system for emitting diffused radiation, comprising:
a radiation source for generating an illuminating beam in a desired wavelength range, and
a beam shaping system, including
a beamsplitting optical element for splitting the illuminating beam into a plurality of beamlets, and
at least one diffusive reflective element for reflecting at least one beamlet with a diffused radiation profile,
wherein the at least one diffusive reflective element is selected from the group comprises one of polytetrafluoroethylene (PTFE) and barium sulphate.

2. The system of claim 1 for emitting diffused radiation, wherein the emitted radiation is in an ultraviolet wavelength range.

3. The system of claim 2 for emitting diffused radiation, wherein the ultraviolet wavelength range is within a 100 nm to 300+ nm germicidal wavelength range.

4. The system of claim 1 for emitting diffused radiation, further comprising:
a treatment chamber for accommodating a flow of one of a liquid and a gas,
wherein the at least one diffusive reflective element is arranged to distribute the diffused radiation profile within a volume of the treatment chamber.

5. The system of claim 4 for emitting diffused radiation, wherein the ultraviolet radiation wavelength is within a 100 nm to 300+ nm germicidal wavelength range.

6. The system of claim 4 for emitting diffused radiation, wherein the liquid is one of water and a food containing water, and the gas is air.

7. The system of claim 4 for emitting diffused radiation, wherein the beamshaping system further includes:
at least one second diffusive reflective for reflecting the at least one beamlet with a diffused radiation profile with a further diffused radiation profile.

8. The system of claim 4 for emitting diffused radiation, wherein the at least one diffusive reflective element for diffusively reflecting the at least one beamlet with a diffused radiation profile comprises at least one interior surface of the treatment chamber.

9. The system of claim 1 for emitting diffused radiation, wherein the radiation source is one of a laser, a solid state light emitting diode and a lamp emitting in the desired wavelength range.

10. The system of claim 1 for emitting diffused radiation, wherein the at least one diffusive reflective element further includes a wavelength shifting additive to allow shifting of the wavelength emitted by the radiation source to wavelengths different form the wavelengths emitted by the radiation source.
